(19) United States
(12) Patent Application Publication
(10) Pub. No.: US 2024/0203581 A1
(43) Pub. Date: Jun. 20, 2024

(54) METHOD OF REMOTELY MANAGING AT LEAST ONE MEDICAL DEVICE OR MEDICAL DEVICE COMPONENT AND REMOTE MANAGEMENT SYSTEM AND MEDICAL DEVICE AND STORAGE SYSTEM

(71) Applicant: OLYMPUS Winter & Ibe GmbH, Hamburg (DE)

(72) Inventors: Thorsten JUERGENS, Hamburg (DE); Andreas MUECKNER, Schwarzenbek (DE); Jakob MUECHER, Hamburg (DE); Andreas NOE, Hamburg (DE); Borg ENDERS, Hamburg (DE); Kathrin TINGELHOFF, Hamburg (DE)

(73) Assignee: OLYMPUS Winter & Ibe GmbH, Hamburg (DE)

(21) Appl. No.: 18/384,983

(22) Filed: Oct. 30, 2023

Related U.S. Application Data
(60) Provisional application No. 63/433,815, filed on Dec. 20, 2022.

(51) Int. Cl. G16H 40/67; G16H 40/40

(52) U.S. Cl. CPC G16H 40/67; G16H 40/40

(57) ABSTRACT

A method of remotely managing at least one medical device or medical device component (hereinafter collectively referred to as “medical device”), wherein a remote management system includes a remote device management server including a device database to store device management data and the medical device including a unique device identifier, the method including: starting the medical device, establishing a communication connection between the medical device and the remote device management server, transmitting the unique device identifier from the medical device to the remote device management server, determining an activation status of the medical device from the device management data, transmitting the activation status from the remote device management server to the medical device, receiving the activation status by the medical device and activating or deactivating the at least one medical device based on the activation status.

### CROSS-REFERENCE TO RELATED APPLICATION

The present application is based upon and claims the benefit of priority from U.S. Provisional Application No. 63/433,815 filed on Dec. 20, 2022, the entire contents of which is incorporated herein by reference.

### BACKGROUND

### Field

The present disclosure relates to a method of remotely managing at least one medical device or medical device component. Furthermore, the present disclosure relates to a remote management system for medical devices or medical device components, such as endoscopes, comprising a remote device management server and at least one medical device or medical device component. The present disclosure also relates to a medical device. Furthermore, the present disclosure relates to a storage system.

### Prior Art

Medical devices and medical device components, such as endoscopes or video endoscopes, are important tools in medical facilities, such as hospitals, doctor's offices and similar places, for addressing the medical needs of patients. Such devices enable or support examinations, surgeries and other treatments.

Medical devices and medical device components often are technologically advanced devices that regularly require service or repair. Such service usually is performed by the manufacturer or another qualified service provider at a place other than the usual location of the medical device, such as a clinic or a medical practice. This requires sending in the medical device to the manufacturer or a service provider.

As the number of medical devices or medical device components at a medical facility is usually limited, the devices available are often in heavy use. However, when sending in the medical equipment for service or repair, it is no longer available for medical diagnostic or surgical procedures in the hospital or other medical facility, thereby creating a problem in the scheduling of such much-needed procedures or creating the need to own expensive replacement units.

This situation is addressed by providing a replacement device for the time of maintenance. As a result, replacement medical devices are sent to, stored and used at various, distributed locations. For this reason, keeping track of and retrieving the medical devices and medical device components in maintenance and in use as replacement devices requires a great deal of administrative effort. The same can be said for loan devices that may be requested by and sent to medical services providing facilities in times of peak demand in order to shore up the limited number of such devices at a location.

### SUMMARY

An object is to increase efficiency of the administration of medical devices that can be stored and used in a distributed manner.

Such object can be solved by a method of remotely managing at least one medical device or medical device component, such as at least one endoscope, wherein a remote management system comprises a remote device management server comprising a device database to store device management data, a processor comprising hardware configured to process the device management data and a server communication unit to communicate with the medical device, wherein the medical device or medical device component comprises a unique device identifier, a device communication unit for communicating with the remote device management server, the method comprising:
a. starting the at least one medical device or medical device component,
b. establishing a communication connection between the at least one medical device or medical device component and the remote device management server,
c. transmitting the unique device identifier from the at least one medical device or medical device component to the remote device management server,
d. determining an activation status of the at least one medical device or medical device component from the device management data, wherein the activation status is “activated” for granting usage or “deactivated” for refusing usage,
e. transmitting the activation status from the remote device management server to the at least one medical device or medical device component, and
f. receiving the activation status by the at least one medical device or medical device component and activating or deactivating the at least one medical device or medical device component based on the activation status.

This method of remotely managing at least one medical device or medical device component considers the following. A medical device or a medical device component is capable of communicating with the remote device management server as a central management unit. The remote device management server collects information about the medical devices and, thus, gathers the device management data. On that basis, the device management server is configured to decide if the usage of a medical device or a medical device component is granted or declined. This decision can be based on various parameters such as, e.g., a duration of use or a limited time permission of use as a replacement device.

Such method can allow for an efficient management of medical devices and medical device components. The device management data for all managed medical devices cam be available at the remote device management server. This way, replacement devices or additional loan devices for demand peaks can be efficiently made available to the user and made to automatically conform to loan or replacement terms by technical means. In a further application, this method can also be used for demonstration units that are loaned to customers or potential customers for a predetermined period of time.

Within the scope of this disclosure, a medical device is medical equipment for multiple use. Such medical equipment can also be referred to as capital equipment. Embodiments of a medical device can be an endoscope, a video endoscope, a defibrillator, medical imaging devices, surgical instruments or further appliances. Medical device components, within the context of this disclosure, refer to components of medical devices. For example, the endoscopic camera head of an endoscope is a medical device component, as is the endoscope that is to be attached to the endoscopic camera head. Another example of a medical device component is the supply unit, such as a camera control unit (CCU), of an endoscope. The corresponding medical device then is the totality of the endoscopic system. These examples for the use of medical devices and medical device components are not to be construed as limiting the scope of medical devices that can be managed with the presently described method, but illustrate the relationship between medical devices and components of medical devices, if such medical devices have several components making up the medical device.

In an embodiment, the unique device identifier of the medial device or the medical device component can be the unique device identification (UDI) as required for medical products. In another embodiment, the unique device identifier can be assigned by the manufacturer. In case of a medical device with multiple components, e.g. a functional unit and a supply unit, the unique device identifier labels in different embodiments the medical device as a whole, or a single medical device component, e.g. the functional unit or the supply unit.

Within the context of the disclosure, the term “remote” refers to a separate device at a distant location. For example, the term “remote” describes a device management server located more distant than a typical supply unit, such as a power supply unit, of a medical device as known in the state of the art. In embodiments, the remote device management server can be located in a different room, a different building, a different city or a different country than the medical device or the medical device component. The communication connection between the at least one medical device or medical device component and the remote device management server can be a bi-directional communication connection. Additionally, or alternatively, there can be a hierarchy of remote servers. For example, one intermediate server in a hospital can serve to bridge the network security to a main server at a central location of the service provider. In that case, the intermediate server can manage the medical device or devices on site and receive the relevant information from the main server.

In an embodiment, the device management data can include operating parameters of the medical device or the medical device component as well as service data. In an embodiment, the device management data can include battery status, light intensity, optical settings, view angle, duration of use, used functions, repair status, service status, software or firmware versions and/or licensing status. As in the case of other listings within the context of this application, this listing is not exhaustive.

In a further embodiment, the at least one medical device or medical device component can collect operating parameters of the at least one medical device or medical device component and can transmit the operating parameters to the device management server. In this embodiment, the device management server can receive the operating parameters and can store the operating parameters in the device database. In an embodiment, the transmitted operating parameters can be included in the device management data.

An activation status of the at least one medical device or medical device component can be determined from the device management data. In an embodiment, the device management data can include data from additional data sources, such as data about at least one of a repair status, a service status, software updates, a security status and a licensing status. Determining an activation status can be advantageous, as necessary maintenance cycles or required software updates or product recalls can be easily transmitted to the device and its user. The licensing status can apply to replacement and loan devices.

In an embodiment, the remote device management server can comprise a computer-based decision support system (CDSS) for supporting in determining the activation status. In an embodiment, the CDSS can be configured to process input parameters by an artificial intelligence (AI) model and to generate a predicted output. In an embodiment, the predicted output can comprise the activation status of the medical device or the medical device component.

In an embodiment, the communication connection between the at least one medical device or medical device component and the device management server can, at least in part, be a wireless communication connection. This can have the advantage that the medical device or medical device component can be more easily usable for the user.

In a further embodiment, the activation status can be “activated” for granting usage or can be “deactivated” for refusing usage or another status of a multitude of other statuses. In an embodiment, a further activation status can be “limited activated”, which can allow the user to use predefined, limited functions of all the functionalities of the medical device or medical device component. In an embodiment, the possibilities of the activation status can include different limited activated status, limiting different functionalities of the medical device. Further activation statuses can hold the advantage of a flexible and efficient management of medical devices and medical device components. With more available statuses, there can be more options than only deactivating the device, when only certain features of the device are affected by a necessary repair, service or software update.

In a further embodiment, the activation status can be determined as “activated”, if a predefined duration of use of the at least one medical device or medical device component is not yet expired or a granted use of the at least one medical device or medical device component has not yet been revoked and/or wherein the activation status can be determined as “deactivated”, if a predefined duration of use of the at least one medical device or medical device component is exceeded or a granted use has been revoked or if the at least one medical device or medical device component is determined to need service or repair. Determining the activation status as activated or as deactivated as described can allow for efficent control and monitoring of the usage of the medical device or the medical device component by the remote device management server. This can also be applicable, if the medical device is a demonstration device that has a predefined usage time period for compliance reasons.

In an embodiment, the predefined duration of use of the at least one medical device or medical device component can be due to an entitlement for usage by renting or licensing the at least one medical device or medical device component. In another embodiment, the predefined duration of use can be given by the duration the at least one medical device or medical component is provided as temporary replacement device for another medical device. In an embodiment, the granted use of an at least one medical device or medical device component can be revoked if a replacement device has arrived at the user. In another embodiment, a granted use can be revoked if a licensing status or a rent status of the at least one medical device or medical device component has expired. In another embodiment, the activation status can be determined as “activated” if other conditions are met. In still another embodiment, the activation status can be determined as “deactivated” if other conditions are met.

In an embodiment, the remote device management server can comprise a CDSS to support in determining if service or repair is needed.

In a further embodiment, the method of remotely managing at least one medical device or medical device component as described above can further comprise determining the location of the at least one medical device or medical device component by the device management server and/or by the at least one medical device or medical device component, comparing the location of the at least one medical device or medical device component with allowed location information in the device management data and setting the activation status of the at least one medical device or medical device component to “deactivated” if the location does not match the allowed location information. This can help in efficiently keeping track of the location of the at least one medical device or medical device component and being able to include the location data into the device management data. This way, the location data of the at least one medical device or medical device component can be available as basis for a decision on the activation status.

In an embodiment, if the location of the at least one medical device or medical device component is determined by the at least one medical device or medical device component, the at least one medical device or medical device component can transmit its location to the remote device management server and the remote device management server can store the location data in a device data base as part of the device management data.

In an embodiment, the remote device management server can comprise a CDSS to support in determining the location of the at least one medical device or medical device component.

In a further embodiment, the at least one medical device or medical device component can comprise a display and/or a video interface, wherein the method can further comprise the at least one medical device or medical device component providing a video output to the display and/or video interface, receiving device management data, such as the activation status, from the remote device management server and including the device management data, such as the activation status, in the video output. Within the scope of the disclosure, the term “video output” refers to any visual visualization, i.e. any graphical output. A video output includes static images, i.e. image data. This embodiment can provide the user fast and reliably with device management data, for example the activation status. The user can be informed that the activation status of the at least one medical device or medical device component is “activated” or “deactivated” or another status.

In an embodiment, the device management data included in the video output can include information such as that the medical device needs to be returned for service, that the medical device and its loan license has expired, or that the activation status is assumed to be changing soon. In another embodiment, the video output can show the remaining time until the activation status is going to be changed from activated to deactivated.

In an embodiment, the display of the medical device or the medical device component can be a display panel on the medical device or the medical device component or can be a user interface on the at least one medical device or medical device component. In another embodiment, the video output can be transmitted to a video interface, which can be configured to be connected to a screen that shows the video output. The device management data or a subset of the device management data can be included in the video output as overlay over the video data. For example, the video output can be video data of an endoscope. The video output can also be shared via a web interface.

Such object can also be solved by a method of remotely managing at least one medical device or medical device component, such as at least one endoscope, such as the method as described above, wherein a remote management system comprises a remote device management server and a storage system, the remote device management server comprising a device database to store device management data, a processor comprising hardware configured for processing the device management data and a server communication unit to communicate with the storage system, wherein the storage system comprises at least one lockable compartment for storing the at least one medical device or medical device component, a computer memory device for storing device storage data about the stored medical device or the stored medical device component, the method comprising establishing a communication connection between the storage system and the remote device management server and locking and/or unlocking the at least one lockable compartment based on locking criteria, wherein the locking criteria are given by an activation status of the at least one medical device or medical device component and/or receiving a remote control command from the remote device management server and/or an input of a passcode and/or a user identification, wherein the activation status is determined from the device management data and/or device storage data, wherein the activation status is “activated” for granting usage or “deactivated” for refusing usage.

This embodiment can provide a storage system, which provides additional medical devices and/or medical device components. It provides these devices on site, e.g. at the medical facility, holding them available on short notice or to be stocked by a supplier on request. If an additional medical device is needed, e.g. because a medical device needs to be repaired unexpectedly or for peak demand, the user can be provided with an additional medical device from the storage system. The storage system can complement the before described “smart” medical device based management logic or replace it, such as in the case of “dumb” medical devices that do not have their own communication units or other prerequisites for the first described version of the method.

In an embodiment, the storage system can be a locker.

The device storage data about the stored medical devices or the stored medical device components can contain similar pieces of information as the device management data on the remote device management server. In embodiments, the device storage data can include data about which medical device or medical device component is stored in which lockable compartment, when the lockable compartment was locked, which user deposited the medical device or medical device component and/or other storage systems specific pieces of information.

In embodiments, the device storage data can be the device management data or a subset thereof, or the device storage data can be an independent data set from the device management data, wherein the device storage data and the device management data can be connectable via the unique device identifier of a medical device or medical device component.

In an embodiment, the storage system can transmit the device storage data or a part of the device storage data to the remote device management server.

In an embodiment, the remote device management server and/or the storage system can comprise a CDSS to support in determining the locking criteria and/or the activation status.

In a further embodiment, the method further can comprise recognizing the at least one medical device or medical device component in a lockable compartment. This can further increase the efficiency of the remote management of medical devices in cases that the lockable compartments are used interchangeably.

In an embodiment, the method can comprise recognizing the at least one medical device or medical device component in the vicinity of a lockable compartment, for example, the recognition can be performed by a scanner in front of the storage system. In another embodiment, the at least one medical device or medical device component can be recognized via a peer-to-peer communication, such as via near-field communication.

In another embodiment, the storage system can comprise a camera and an image processor comprising hardware for recording a unique device identifier and/or a serial number of a medical device or medical device component. The at least one medical device or medical device component can comprise a barcode with its unique device identifier and/or can comprise a barcode with its serial number and can be recognized by this barcode.

In an embodiment, the remote device management server and/or the storage system can comprise a CDSS to support in recognizing the at least one medical device or medical device component.

Such object can also be solved by a remote management system for at least one medical device or medical device component, such as at least one endoscope, comprising a remote device management server and at least one medical device or medical device component, the at least one medical device or medical device component having a unique device identifier and a device communication unit for communicating with the remote device management server, the remote device management server comprising a device database to store device management data, a processor comprising hardware configured for processing the device management data and a server communication unit configured to communicate with the at least one medical device or medical device component, wherein the device communication unit is configured to establish a communication connection to the remote device management server and the at least one medical device or medical device component is configured to transmit the unique device identifier to the remote device management server, wherein the remote device management server is configured to determine a remote control command for remotely managing the at least one medical device or medical device component and to transmit the remote control command to the at least one medical device or medical device component, wherein the at least one medical device or medical device component is configured to receive and execute the remote control command from the remote device management server, wherein the remote management system can be configured to perform a method of remotely managing at least one medical device or medical device component as described above.

Such a remote management system can provide for an efficient administration of medical devices stored and used in a distributed manner. The remote device management server can keep track of the device management data and can be configured, if necessary, to transmit remote commands to the at least one medical device or medical device component. This way, optimal performance of the medical devices and low down-times with a reduced number of medical devices can be achieved.

In an embodiment, the remote management system as described above can be a loan system. It allows for remotely controlled loaning of medical devices such as endoscopes as well as medical device components.

In an embodiment, the remote device management server can be a server computer including a central processing unit (CPU) as the processor. In another embodiment, the remote device management server can be a cloud computer system.

In an embodiment, the remote device management server can be configured to determine the remote control command based on the device management data. This can allow suitable remote commands for the corresponding medical device or medical device component.

In an embodiment, the remote device management server can be configured to determine a remote control command, wherein the remote control command can activate the device, disabling the device, disabling or limiting a specific functionality of the device, enabling a specific functionality of the device, activating or deactivating energy saving mode, starting software update, and/or displaying device management data in a video output. The remote control commands can include further remote control commands, such as suitable to the at least one medical device or medical device component.

In another embodiment, the at least one medical device or medical device component can be configured to collect operating parameters of the at least one medical device or medical device component and can transmit the operating parameters to the remote device management server, wherein the remote device management server can be configured to receive the operating parameters and to store the operating parameters in the device database. The operating parameters can include, without limiting the scope of operating parameters, battery status, light intensity, optical settings, view angle, duration of use, used functionalities and/or software status and/or other parameters.

In a further embodiment, the at least one medical device or medical device component of the remote management system as described above can comprise a functional unit for performing a medical procedure and a supply unit for supplying the functional unit, wherein the supply unit can comprise a power supply unit supplying power to the functional unit and a controller comprising hardware configured to control the functional unit, wherein the functional unit or the supply unit can comprise the device communication unit. Separating a medical device or a medical device component in two different units can hold the advantage of more efficient workflows and better specialization of the different hardware devices.

For example, an endoscope can be the functional unit of a medical device and the camera control unit (CCU) can act as supply unit of the medical device.

In an embodiment, the power supply can be a permanent power supply unit, while in another embodiment the power supply unit can recharge a battery of the functional unit.

In an embodiment with a functional unit and a supply unit of a medical device or a medical device component, the unique device identifier can identify the functional unit or the supply unit or the combination of functional unit and supply unit. The device communication unit can be part of the supply unit or can be part of the functional unit.

Controlling the functional unit can include setting operating parameters, such as light intensity and/or view angle and/or camera settings and/or processing and or displaying sensor data obtained by sensors, transmitting and/or processing data, displaying data, such as on a screen or a display.

In a further embodiment, the communication connection to the remote device management server can be a wireless communication connection, such as based on a cellular network and/or based on a low-power wide-area network. This can hold the advantage of a stable and independent communication connection between the at least one medical device or medical device component and the remote device management server.

In another embodiment, the communication connection to the remote device management server can be a wireless communication connection based on a wireless local area network. In another embodiment, the communication connection to the remote device management server can be a wire-bound communication connection, such as based on Ethernet. In still another embodiment, the communication connection to the remote device management server can in part be a wireless communication connection.

The wireless communication connection can be based on a cellular network and/or a low-power wide-area network, such as based on at least one of LTE, 5G, LTE Machine Type Communication (LTE-M), Narrowband Internet of things (NB-IOT), LoRa, LoRaWAN, Sigfox and MIOTY.

In a further embodiment, the device communication unit can comprise an energy storage element, such as a battery, and the device communication unit can be configured to operate by being powered by the energy storage element. This embodiment can be advantageous, if the device communication unit is operating via cellular network and/or low-power wide-area network. It can enable the medical device or the medical device component to communicate with the remote device management server without power supply from the user.

In a further embodiment, the remote device management server can be configured to determine the location of the at least one medical device or medical device component and/or wherein the at least one medical device or medical device component can comprise a position locator unit for determining position data of the at least one medical device or medical device component, wherein the at least one medical device or medical device component can be configured to transmit the position data to the remote device management server. Identifying the position of the at least one medical device or medical device component can add an additional criterion for determining remote control commands to be sent to the at least one medical device or medical device component.

Within the context of this disclosure, the location of the at least one medical device or medical device component can refer to the spatial location of the device. The term position data can refer to the location of a medical device or medical device component and/or secondary location information, such as the information about telecommunication networks as network data and receptor strength.

In an embodiment, the remote device management server can determine the location of the at least one medical device or medical device component by properties of the communication, such as the IP address, network identifiers, particular network notes, geographic information and/or static IP addresses. In another embodiment, the communication can be authenticated by a user and a location passcode.

In an embodiment, the at least one medical device or medical device component can be configured to transmit network data about detected networks, such as cellular network information and/or wireless local area network information, to the remote device management server. Then, the remote device management server can be configured to analyze the network data obtained from the at least one medical device or medical device component, and locate the position of the medical device.

The position locator unit of the at least one medical device or medical device component can be a GPS module and can be configured to determine the position data by GPS. Alternatively, the device communication unit can comprise the position locator unit. In an embodiment, the position locator unit can be configured to determine the position data based on information derived from a cellular network and/or based on the receptor strength of other communication networks, such as wireless local area networks.

The position locator unit can be located in the handle area of the medical device or the medical device component or in the connector area of an endoscope. Alternatively, the position locator unit can be integrated into the electronics or into the printed circuit board of the electronic sub-assembly.

In a further embodiment, the at least one medical device or medical device component can comprise a display and/or a video interface and can be configured to provide a video output, wherein the at least one medical device or medical device component can be configured to receive device management data from the remote device management server and to include the device management data in the video output. Through this, the user can be easily informed about the device management data on a regular basis, such as duration of usage, remaining duration of usage, or activation status. By integrating this information into the video output of the medical device or medical device component, it is directly on display for the user. By displaying the device management data on a separate display, it simplifies using the medical device for the user.

In an embodiment, wherein the medical device or the medical device component comprises a functional unit and a supply unit, the functional unit can comprise the display and/or the video interface or the supply unit can comprise the display and/or the video interface. For example, the medical device can be an endoscope with an integrated image sensor, such as an endoscope with chip-at-the-tip technology and/or an endoscope with an image sensor in the handle, and the medical device can include a display. In another example, the medical device can be an endoscope system, wherein the endoscope system can comprise an endoscope head as a functional unit comprising an optical system without a sensor chip and a supply unit comprising a sensor chip for processing the optically transmitted video data. In this example, the supply unit can comprise the display and/or the video interface to provide the user with the device management data.

In a further embodiment, the remote control command transmitted by the remote device management server can be a remote control command to activate or deactivate the at least one medical device or medical device component, wherein the remote control command to activate or to deactivate the at least one medical device or medical device component can be based on an activation status of the at least one medical device, wherein the remote device management server can be configured to determine the activation status of the at least one medical device or medical device component on the basis of the device management data of the at least one medical device or medical device component. This can hold the advantage that in case of a necessary repair or maintenance or in case of an exceeded duration of usage, the medical device or medical device component can be easily disabled, e.g. for being returned to the manufacturer. Determining the activation status on basis of the device management data can allow for an efficient remote management of the medical devices.

In an embodiment, an additional remote control command can be a remote control command to enable predefined features or disable predefined features of the at least one medical device or medical device component. Another remote control command can start a timer on the medical device or medical device component and can activate or can deactivate the medical device or medical device component when the timer is run down.

In another embodiment, the activation status can be “activated” for granting usage or “deactivated” for refusing usage. There can be additional activation status, such as “limited activated” for activating the medical device or medical device component for only a predefined limited set of features. The activation status can comprise dates and/or durations of usage until an activation and/or deactivation event. This can be useful if the communication connection between the medical device or medical device component and the remote device management server is not always available.

In a further embodiment, the remote management system can comprise a storage system, wherein the storage system can comprise at least one lockable compartment for storing at least one medical device or medical device component, a computer memory device for storing device storage data about the stored medical device or stored medical device component, and a storage communication unit configured to communicate with the remote device management server and to transmit the device storage data to the remote device management server, wherein the storage system can be configured to lock or unlock the at least one lockable compartment based on locking criteria, such as on the activation status of the at least one medical device or medical device component and/or on receiving a remote control command from the remote device management server and/or on an input of a passcode and/or on a user identification. A storage system can yield the advantage of a simple access for issuing and taking back medical devices and medical device components, which can be intended for interim use, e.g. for limited usage times in a loan system.

In an embodiment, the remote management system can be configured to perform a method including a storage system as described above.

In an embodiment, the storage system can be configured to receive remote control commands from the device management server. The remote control command can be a command to unlock a predefined compartment or to unlock a compartment containing a predefined medical device or medical device component. Another remote control command can be to lock a predefined compartment. The storage system can be configured to lock a lockable compartment after a user has inserted a medical device or a medical device component into the compartment. Also, the storage system can be configured to lock unlocked compartments automatically after a predefined period of time.

In an embodiment, the storage communication unit can be configured to communicate via a wireless connection with the remote device management server. For example, the wireless connection can be a cellular network and/or a lowpower wide area network, such as at least one of LTE, 5G, LTE Machine Type Communication (LTE-M), Narrowband Internet of things (NB-IOT), LoRa, LoRaWAN, Sigfox and MIOTY. The communication connection between storage system and the remote device management server can be in part wireless and in part wire-bound.

In a further embodiment, the storage system can comprise a medical device recognition unit, such as comprising a camera and an image processor comprising hardware and/or a near-field communication sensor, configured to recognize at least one medical device and/or medical device component in a lockable compartment. This can allow for a simple and efficient identification of medical devices and medical device components in the storage system.

In an embodiment, the medical device recognition unit can comprise a peer-to-peer communication device.

For being recognized by the image processor, the at least one medical device or medical device component can comprise a bar code with the unique device identifier or can comprise a serial number attached or printed on the surface of the medical device or medical device component. In a corresponding embodiment, a camera and an image processor comprising hardware can be configured to record a unique device identifier and/or serial number of a medical device as described above.

Such object can also be solved by a medical device of a remote management system as described above.

Such object can be furthermore solved by a medical device component of a remote management system as described above.

Such object can also be solved by a storage system of a remote management system as described above.

The above-described method and remote management system and its components, including the medical device, the medical device component and the storage system, constitute several embodiments. The system and its constituents are set up to incorporate the above-described method with all its embodiments, and the method can be incorporated using the above-described components of the system.

### BRIEF DESCRIPTION OF THE DRAWINGS

Further characteristics will become apparent from the description of the embodiments together with the claims and the included drawings. Embodiments can fulfill individual characteristics or a combination of several characteristics.

The embodiments are described below, without restricting the general intent of the invention, based on the exemplary embodiments, wherein reference is made expressly to the drawings with regard to the disclosure of all details that are not explained in greater detail in the text. In the drawings:

FIG. 1 illustrates a schematic view of a first embodiment of a remote management system including a first embodiment of a medical device and a first embodiment of a remote device management server,

FIG. 2 illustrates a schematic view of a second embodiment of a medical device,

FIG. 3 illustrates a schematic view of an embodiment of a medical device component,

FIG. 4 illustrates a schematic front view of a first embodiment of a storage system,

FIG. 5 illustrates a schematic front view of the storage system in FIG. 4 with an opened lockable compartment,

FIG. 6 illustrates a schematic view of the steps of a first embodiment of a method of remotely managing at least one medical device or medical device component,

FIG. 7 illustrates a schematic view of the steps of a second embodiment of a method of remotely managing at least one medical device or medical device component, and

FIG. 8 illustrates a schematic view of steps performed by a computerbased decision support system.

In the drawings, the same or similar types of elements or respectively corresponding parts are provided with the same reference numbers in order to prevent the item from needing to be reintroduced.

### DETAILED DESCRIPTION

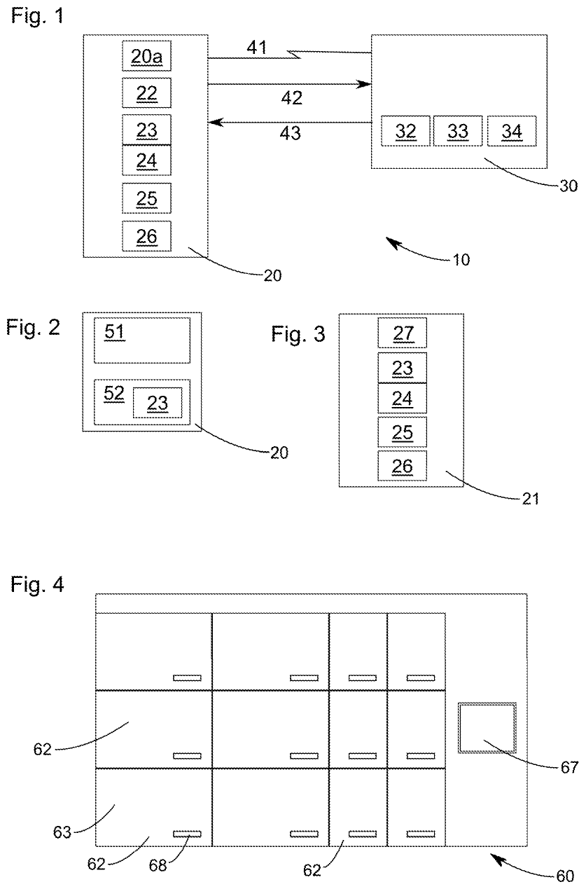

FIG. 1 shows a schematic view of a first embodiment of a remote management system 10 including a first embodiment of a medical device 20 and a first embodiment of a remote device management server 30. In this embodiment, the medical device 20 is a video endoscope. It comprises a controller 20a and a device communication unit 23 (i.e., transceiver), which may be configured to communicate via SIGFOX as low-power wide-area network. Furthermore, the medical device 20 may comprise a battery 24 to power the device communication unit 23. The medical device 20 comprises a memory 25 having a unique device identifier that is assigned by the manufacturer and stored electronically on the medical device 20, which may be implemented in the controller 20a of medical device 2. Moreover, the medical device 20 comprises a position locator unit 26, e.g., a GPS module (GPS Receiver). The medical device 20, or the controller 20a of the medical device, further comprises a display 22 to display operating parameters or the activation status of the medical device 20 to the user. Controller 20a can be configured as dedicated hardware circuits or a hardware processor executing a series of software instructions.

In this embodiment, the device management server 30 is a server computer comprising a device database 32, a processor 33, which is the central processing unit (CPU) of the server computer 30, as well as a server communication unit 34 (i.e., transceiver). Processor 33 can be configured as dedicated hardware circuits or a hardware processor executing a series of software instructions.

The device communication unit 23 and the server communication unit 34 enable the medical device 20 and the remote device management server 30 to communicate via a communication connection 41. In this embodiment, this is a wireless communication connection via SIGFOX. Via this communication connection, the medical device 20 is configured to transmit the unique device identifier 25 via a unique device identifier signal 42 to the remote device management server 30. The remote device management server 30 processes the unique device identifier 25 and determines on the basis of the device management data stored in the device database 32 a remote control command 43 to be executed by the medical device 20. The remote control command 43 is transmitted from the remote device management server 30 to the medical device 20 and executed by the medical device 20.

In this embodiment, the remote control commands 43 are “activate”, “deactivate”, “activate with limited functions” and “update display”. While “activate” activates the medical device 20 and permits usage, “deactivate” deactivates the medical device 20 and prohibits usage and “activate with limited functions” only allows for video endoscope with limited settings, e.g. certain light intensities, view angles, or measurement modes. The remote control command 43 “update display” makes the medical device 20 update the display 22 with the latest activation status.

FIG. 2 shows a schematic view of a second embodiment of a medical device 20. In this embodiment, the medical device 20 is a video endoscope comprising a functional unit 51 and a supply unit 52. The supply unit 52 supplies the functional unit 51 with power and receives video data obtained by the functional unit 51. The functional unit 51 comprises an optical system including lenses and an imaging sensor, while the supply unit 52 receives the video data acquired by the imaging sensor, performs video data processing and shows a video output to the user. In this embodiment, the supply unit 52 comprises the device communication unit 23 (i.e., transceiver). In this embodiment, the device communication unit 23 comprises an Ethernet interface for wire-bound communication. In this embodiment, the medical device 20 does not comprise a battery 24 for supplying power to the device communication unit 23. Supply unit 52 can be configured as dedicated hardware circuits or a hardware processor executing a series of software instructions.

The functional unit 51 may also comprise the device communication unit 23 (i.e., transceiver) akin to the embodiment displayed in FIG. 1, comprising an interface for communication via a LTE-M as cellular network, for example, as well as a battery 24 for the device communication unit 23.

In FIG. 3, a medical device component 21 is shown that is designed to be added to medical devices that cannot, by themselves, be remotely managed. Such a medical device component 21 may be an adapter that can be attached to the medical device, for example to its handle.

The medical device component 21 as shown in FIG. 3 comprises a device communication unit 23 (i.e., transceiver), a battery 24 and a memory 25 storing the unique device identifier 25, wherein the unique device identifier 25 is stored electronically on the memory comprised by the medical device component 21 and is stored in a bar code attached to the surface of the medical device component 21. Furthermore, the medical device component 21 comprises a position locator unit 26, which is a wireless communication unit that may, for example, detect the receptor strength of wireless local area networks or cellular networks in order to acquire position data of the medical device component 21. In this embodiment, the position data acquired by the position locator unit 26 are transmitted by the device communication unit 23 to the remote device management server 30 that uses a CDSS to support identifying the position of the medical device component 21 from the transmitted data. The video interface 27 of the medical device component 21 may be a USB-C interface.

FIG. 4 shows a schematic front view of a first embodiment of a storage system 60. In this embodiment, the storage system 60 is a locker. It comprises multiple lockable compartments 62 of different sizes. Each compartment is closed by a lockable door 63 and can be opened via a handle 68, if the corresponding lockable compartment 62 is unlocked. Alternatively, the unlocking action may open the respective door 63. Locks having wired or wireless actuators for unlocking a door are well-known in the art. The storage system 60 further comprises a user interface 67, which a user can use for requesting a medical device 20 or a medical device component 21 from the storage system 60 or processing the return of a medical device 20 or a medical device component 21 into the storage system 60.

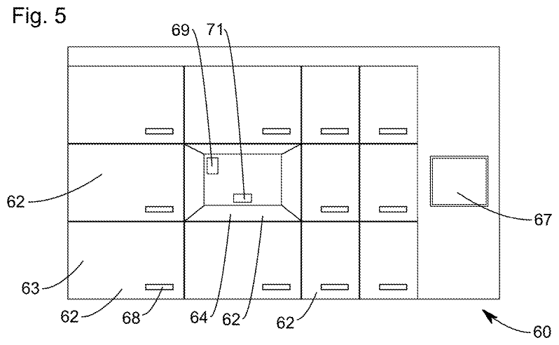

FIG. 5 shows a schematic front view of the storage system in FIG. 4 with an opened lockable compartment 62 with its interior 64. The door 63 of the opened lockable compartment 62 is omitted in the drawing for clarity's sake. In the interior 64 of the opened lockable compartment 62, two medical device recognition units are located. One of them, a camera 69 is configured, together with an image processor not shown in FIG. 4, to recognize medical devices 20 or medical device components 21 inserted into the opened lockable compartment 62. When recognized, the medical device recognition unit transmits the data to the storage system 60 to be stored with the device storage data. As a second medical device recognition unit, a near-field communication sensor 71 is integrated in the interior 64 of the lockable compartment 62. It is configured to recognize a medical device 20 or medical device component 21 inserted into the interior 64 of the lockable compartment 62, wherein the medical device 20 or medical device component 21 is equipped with a near-field communication tag.

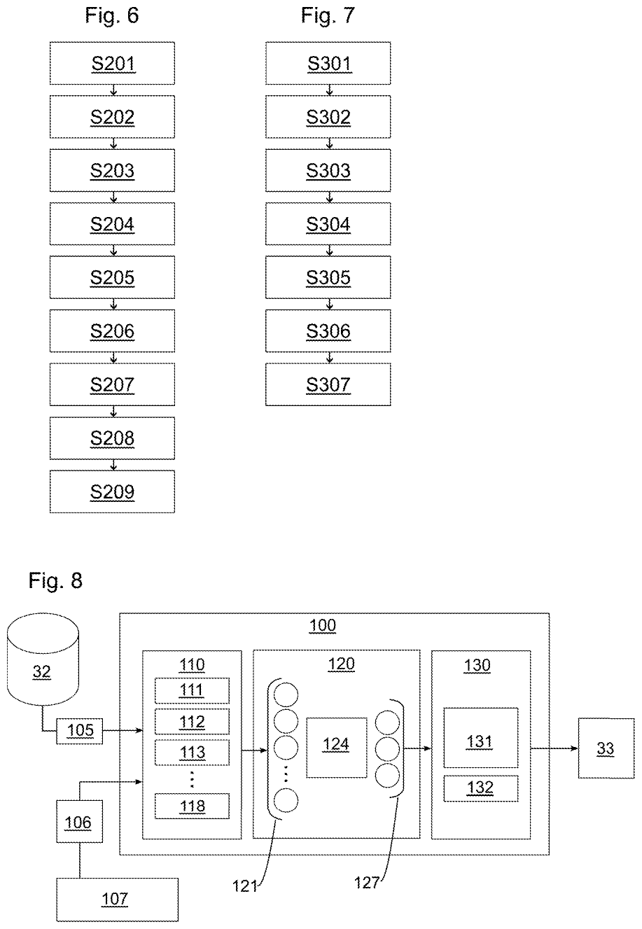

FIG. 6 shows a schematic view of the steps of a first embodiment of a method of remotely managing at least one medical device or medical device component. In a first step S201, a medical device 20 is started up. Here, the medical device 20 is an endoscope and starting up refers to switching on the device in order to prepare for a treatment of a patient. Subsequently, the method comprises establishing a communication connection between the medical device 20 and the remote device management server 30 in step S202. Subsequently, the medical device 20 determines its location by a built-in GPS module (step S203). In the next step S204, the medical device 20 transmits the location together with the unique device identifier 25 to the remote device management server 30.

Subsequently, in step S205, the remote device management server 30 determines the activation status of the medical device 20. For this, the remote device management server 30 processes the device management data from the device database 32. In this embodiment, for the unique device identifier 25 of this medical device 20 the device management data may contain a data set of “end date usage”. The remote device management server 30 compares this date with the current date and may find the end date of usage to be two days in the past. Consequently, the remote device management server 30 determines the predefined duration of use of the medical device 20 to be exceeded and determines the activation status to be “deactivated”.

In the next step S206, the remote device management server 30 transmits the activation status determined in the previous step S205 to the medical device 20. Subsequently, in step S207, it receives the activation status and the medical device 20 deactivates itself (step S208). In a final step S209, the medical device 20 provides video output informing the user of the exceeded duration of usage in a display of the medical device 20.

In another embodiment, the remote device management server 30 determines the activation status in a step similar to step S205. In this other embodiment, the device management data leads the remote device management server 30 to determine an activation status of “activated” for the medical device 20. In the following steps, the medical device 20 is activated, which is also shown in the video output.

FIG. 7 shows a schematic view of the steps of a second embodiment of a method of remotely managing at least one medical device or medical device component. This method comprises the use of a storage system 60.

In a first step S301, a user requests the usage of a medical device at a storage system 60. For this, the user authenticates herself or himself with a user authentication passcode at the storage system 60. Subsequently, the storage system 60 establishes a communication connection between the remote device management server 30 and the storage system 60 (step S302). The storage system 60 transmits the request of the user together with a subset of the device storage data, in this embodiment the current occupancy of the lockable compartments 62, to the remote device management server 30 (step S303).

Next, in step S304, the remote device management server 30 receives the request and determines the activation status of one or more medical devices 20 that are suitable for the user's request, e.g. a particular type of medical device with requested functionality. In this embodiment, the remote device management server 30 determines the activation status of the medical device 20 to be “activated” and transmits this activation status together with information about the medical device 20, such as its unique device identifier 25, to the storage system 60 (step S305).

In the following step S306, the storage system 60 receives the activation status of the medical device 20 and identifies the corresponding lockable compartment 62 of the storage system 60. In the next step S307, the storage system 60 unlocks the corresponding lockable compartment. The user can withdraw the medical device 20 as requested from the storage system 60.

In another embodiment, not shown in the figures, a user requests to return a medical device 20 to a storage system 60. In a similar fashion as described for FIG. 7, the storage system 60 transmits this request to the remote device management server 30 that processes the request, updates the device management data in the device database 32, and transmits a remote control command to the storage system 60. In this embodiment, the device management server updates the activation status of the medical device 20 to “deactivated”. The remote control command triggers the storage system to unlock a suitable lockable compartment 62, so that the user can insert the medical device 20 into the interior 64 of the lockable compartment 62, and triggers the storage system 60 to lock the lockable compartment 62 subsequently. With this transaction, the user has successfully returned a medical device 20, which is afterwards locked in the storage system 60, and the device management data is updated accordingly.

FIG. 8 shows a schematic view of steps performed by a computer-based decision support system (CDSS) 100 that is configured to determine the position of at least one medical device or medical device component, activation status, and/or remote control commands to be executed by the medical device or medical device component based on input parameters 121. In various embodiments, the CDSS 100 includes an input interface 110 through which input parameters based upon the device management data 105 and additional input data 106 from additional input data sources 107 which are specific to a medical device or medical device component are provided as input features to an artificial intelligence (AI) model 120, a processor which performs an inference operation in which the input parameters are applied to the AI model to generate a predicted output 131, such as the position and/or activation status of the medical device or medical device component and/or remote control commands to be executed by the medical device or medical device component and an output interface 130 through which the predicted output 131, such as including confidence score(s) 132, is communicated to the processor 33 of the remote device management server 30.

In some embodiments, the input interface 110 may be a direct data link between the CDSS 100 and one or more data sources 32, 107 that generate at least some of the input features. For example, the input interface 110 may transmit the unique device identifier and/or duration of usage and/or position within the storage system directly to the CDSS when requesting the activation of a medical device or medical device component. Additionally, or alternatively, the input interface 110 may be a classical user interface that facilitates interaction between a user and the CDSS 100. For example, the input interface 110 may facilitate a user interface through which the user may manually enter a unique device identifier and/or user identification and/or an activation code. Additionally, or alternatively, the input interface 110 may provide the CDSS 100 with access to the device database 32 providing the device management data 105 from which one or more input features may be extracted. In any of these cases, the input interface 110 is configured to collect one or more of the following input features in association with a specific medical device or medical device component on or before a time at which the CDSS 100 is used to assess the activation status, the position of and/or remote control commands for or other information on the medical device or medical device component. The input features (111, 112, 113, . . . , 118) may be parameters such as duration of use, repair status, last maintenance date, licensing status, loan status, software version, battery status, light intensity, optical settings, view angles. Further input parameters 106 from additional data sources 107 may include information about other medical devices, such as known manufacturing defects, release dates of software updates, parameters of typical usage and necessary repair and maintenance cycles. In other embodiments, the input interface 110 of the CDSS 100 furthermore includes other input parameters.

Based on one or more of the above input features, the processor performs an inference operation using the AI model 120 to generate the predicted output 131. For example, input interface 110 may deliver the input parameters 121 of duration of usage, last maintenance date and current date into an input layer 121 of the AI model 120, which propagates these input features through the AI model 120 to an output layer 127. The AI model 120 can provide a computer system the ability to perform tasks, without explicitly being programmed, by making inferences based on patterns found in the analysis of data. AI model 120 explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), NaiveBayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model 120 may be trained continuously or periodically prior to performance of the inference operation by the processor. Then, during the inference operation, the input features specific to the medical device or medical device component may be provided to the AI model 120 and may be propagated from an input layer 121, through one or more hidden layers 124, and ultimately to an output layer 127 that corresponds to the predicted output 131. For example, if various medical devices of a specific model type have shown to require maintenance or repair, this information is included for the assessment of the activation status and/or the necessity of a maintenance or repair of another medical device of this specific model type.

During and/or subsequent to the inference operation, the predicted output 131 may be communicated to the user via a user interface of the medical device or medical device component and/or automatically be transmitted to the remote device management server 30 for adjusting the device database 32 and performing the subsequent steps of action. For example, if the CDSS determines that the activation status of a medical device or medical device component is set to “deactivated” due to an exceeded duration of usage time or an expected failure due to missing maintenance, the remote device management server 30 transmits a remote command to deactivate the corresponding medical device. In another example, the CDSS determines the activation status of a medical device component stored in a storage system to be “activated”, the remote device management server 30 transmits this information to the storage system 60 and the storage system 60 unlocks the corresponding lockable compartment 62.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### List of References

20 medical device
20a controller
21 medical device component
22 display
23 device communication unit
24 battery
25 memory
26 position locator unit
27 video interface
30 remote device management server
32 device database
33 processor
34 server communication unit
41 communication connection
42 unique device identifier signal
43 remote control command
51 functional unit
52 supply unit
60 storage system
62 lockable compartment
63 door
64 interior
65 computer memory device
66 storage communication unit
67 user interface
68 handle
69 camera
70 near-field communication sensor
100 computer-based decision support system (CDSS)
105 device management data
106 additional input data
107 additional input data source(s)
110 input interface
111 first input feature
112 second input feature
113 third input feature
118 n-th input feature
120 AI model
121 input layer
124 hidden layers
127 output layer
130 output interface
131 predicted output
132 confidence score(s)
S201 starting up
S202 establishing a communication connection
S203 determining the location
S204 transmitting the unique device identifier and the location
S205 determining an activation status
S206 transmitting the activation status
S207 receiving the activation status

S208 activating or deactivating the at least one medical device or medical device component
S209 providing video output
S301 requesting usage of a medical device
S302 establishing a communication connection
S303 transmitting the request
S304 determining an activation status
S305 transmitting an activation status
S306 receiving activation status
S307 unlocking compartment

What is claimed is:

1. A method of remotely managing at least one medical device or medical device component, wherein a remote management system comprises a remote device management server comprising a device database to store device management data, a processor comprising hardware configured for processing the device management data and a server communication unit to communicate with the at least one medical device, wherein the at least one medical device or medical device component comprises a memory storing a unique device identifier, a device communication unit for communicating with the remote device management server, the method comprising:
a. starting the at least one medical device or medical device component,
b. establishing a communication connection between the at least one medical device or medical device component and the remote device management server,
c. transmitting the unique device identifier from the at least one medical device or medical device component to the remote device management server,
d. determining an activation status of the at least one medical device or medical device component from the device management data, wherein the activation status is “activated” for granting usage or “deactivated” for refusing usage,
e. transmitting the activation status from the remote device management server to the at least one medical device or medical device component, and
f. receving the activation status by the at least one medical device or medical device component and activating or deactivating the at least one medical device or medical device component based on the activation status.

2. The method according to claim 1, wherein:
the activation status is determined as “activated”, if a predefined duration of use of the at least one medical device or medical device component is not yet expired or a granted use of the at least one medical device or medical device component has not yet been revoked; or the activation status is determined as “deactivated”, if a predefined duration of use of the at least one medical device or medical device component is exceeded or a granted use has been revoked or if the at least one medical device or medical device component is determined to need service or repair.

3. The method according to claim 1, further comprising:
determining the location of the at least one medical device or medical device component by one of the device management server and by the at least one medical device or medical device component,
comparing the location of the at least one medical device or medical device component with allowed location information in the device management data; and
setting the activation status of the at least one medical device or medical device component to “deactivated” if the location does not match the allowed location information.

4. The method according to claim 1, wherein the at least one medical device or medical device component comprises a display and/or a video interface, wherein the method further comprises the at least one medical device or medical device component providing a video output to the display and/or video interface, receiving device management datafrom the remote device management server and including the device management data in the video output.

5. The method according to claim 1, wherein a remote management system comprises a remote device management server and a storage system, the remote device management server comprising a device database to store device management data, a processor comprising hardware configured for processing the device management data and a server communication unit to communicate with the storage system, wherein the storage system comprises at least one lockable compartment for storing the at least one medical device or medical device component, a computer memory for storing device storage data about the stored medical device or the stored medical device component, the method comprising:
establishing a communication connection between the storage system and the remote device management server and locking and/or unlocking the at least one lockable compartment based on locking criteria, wherein the locking criteria are given by one of an activation status of the at least one medical device or medical device component, receiving a remote control command from the remote device management server, an input of a passcode, a user identification, wherein the activation status is determined from the device management data, and device storage data, wherein the activation status is “activated” for granting usage or “deactivated” for refusing usage.

6. The method according to claim 5, further comprising recognizing the at least one medical device or medical device component in a lockable compartment.

7. A remote management system for at least one medical device or medical device component, comprising a remote device management server and at least one medical device or medical device component, the at least one medical device or medical device component having a memory storing a unique device identifier and a device communication unit for communicating with the remote device management server, the remote device management server comprising a device database to store device management data, a processor comprising hardware configured for processing the device management data and a server communication unit configured to communicate with the at least one medical device or medical device component,
wherein the device communication unit is configured to establish a communication connection to the remote device management server and the at least one medical device or medical device component is configured to transmit the unique device identifier to the remote device management server,
wherein the remote device management server is configured to determine a remote control command for remotely managing the at least one medical device or medical device component and to transmit the remote control command to the at least one medical device or medical device component,
wherein the at least one medical device or medical device component is configured to receive and execute the remote control command from the remote device management server, and
wherein the remote management system is configured to perform a method of remotely managing at least one medical device or medical device component according to claim 1.

8. The remote management system according to claim 7, wherein the at least one medical device or medical device component comprises a functional unit for performing a medical procedure and a supply unit for supplying the functional unit, wherein the supply unit comprises a power supply supplying power to the functional unit and a controller comprising hardware configured to control the functional unit, wherein the functional unit or the supply unit comprises the device communication unit.

9. The remote management system according to claim 7, wherein the communication connection to the remote device management server is a wireless communication connection.

10. The remote management system according to claim 7, wherein the device communication unit comprises an energy storage element, and the device communication unit is configured to operate by being powered by the energy storage element.

11. The remote management system according to claim 7, wherein one of the remote device management server is configured to determine the location of the at least one medical device or medical device component or wherein the at least one medical device or medical device component comprises a position locator unit for determining position data of the at least one medical device or medical device component, wherein the at least one medical device or medical device component is configured to transmit the position data to the remote device management server.

12. The remote management system according to claim 7, wherein the at least one medical device or medical device component comprises one of a display or a video interface configured to provide a video output, wherein the at least one medical device or medical device component is configured to receive device management data from the remote device management server and to include the device management data in the video output.

13. The remote management system according to claim 7, wherein the remote control command is a remote control command to activate or deactivate the at least one medical device or medical device component.

14. The remote management system according to claim 13, wherein the remote control command to activate or to deactivate the at least one medical device or medical device component is based on an activation status of the at least one medical device, and the remote device management server is configured to determine the activation status of the at least one medical device or medical device component on the basis of the device management data of the at least one medical device or medical device component.

15. The remote management system according to claim 7, further comprising a storage system, wherein the storage system comprises at least one lockable compartment for storing at least one medical device or medical device component, a computer memory device for storing device storage data about the stored medical device or stored medical device component, and a storage communication unit configured to communicate with the remote device management server and to transmit the device storage data to the remote device management server,
wherein the storage system is configured to lock or unlock the at least one lockable compartment based on locking criteria.

16. The remote management system according to claim 15, wherein the storage system is configured to lock or unlock the at least one lockable compartment based on locking criteria on one or more of the activation status of the at least one medical device or medical device component, on receiving a remote control command from the remote device management server and on an input of a passcode and/or on a user identification.

17. The remote management system according to claim 14, wherein the storage system comprises a medical device recognition unit configured to recognize at least one medical device and/or medical device component in a lockable compartment.

18. The remote management system according to claim 14, whereinthe medical device recognition unit comprises one of a camera and an image processor comprising hardware and a near-flied communciation sensor.

19. A medical device of a remote management system according to claims 7.

20. A storage system of a remote management system according to claim 14.
